(19) United States
(12) Patent Application Publication
(10) Pub. No.: US 2013/0317451 A1
(43) Pub. Date: Nov. 28, 2013

(54) THREADED LOCK SYSTEM

(71) Applicant: Lipovera, LLC, North Attleboro, MA (US)

(72) Inventor: Daniel A. Del Vecchio, Wrentham, MA (US)

(73) Assignee: Lipovera, LLC, North Attleboro, MA (US)

(21) Appl. No.: 13/900,086

(22) Filed: May 22, 2013

Related U.S. Application Data
(60) Provisional application No. 61/650,577, filed on May 23, 2012.

(51) Int. Cl. A61M 5/34

(52) U.S. Cl. CPC A61M 5/345
USPC 604/241

(57) ABSTRACT

A syringe cap or needle attachment includes an interior coupling region with sharp, rigid spirals to thread soft plastic syringe ends during attachment and form a secure mechanical fit.

### RELATED APPLICATIONS

This application claims the benefit of U.S. App. No. 61/650,577, filed on May 23, 2012, the entire content of which is hereby incorporated by reference.

### BACKGROUND

Luer lock style syringes provide mating threads for secure, twist-lock attachment to needles and the like. Other common syringes, however, do not have threads or other positive locking mechanisms and rely on friction fits or external clamps for secure engagement. There remains a need for secure coupling to unthreaded syringes.

### SUMMARY

A syringe cap or needle attachment includes an interior coupling region with sharp, rigid spirals to thread soft plastic syringe ends during attachment and form a secure mechanical fit.

### BRIEF DESCRIPTION OF THE DRAWINGS

The invention and the following detailed description of certain embodiments thereof may be understood by reference to the following figures:

FIG. 1 shows a threaded cap.

FIG. 2 shows a threaded connector.

FIG. 3 shows a needle with a threaded attachment.

### DETAILED DESCRIPTION

All documents mentioned herein are hereby incorporated in their entirety by reference. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Thus the term “or” should generally be understood to mean “and/or” and so forth.

Disclosed herein is a threaded cap or connector with internal threads that thread and attach to an unthreaded end of a syringe or the like in order to lock the cap/connector securely to the syringe.

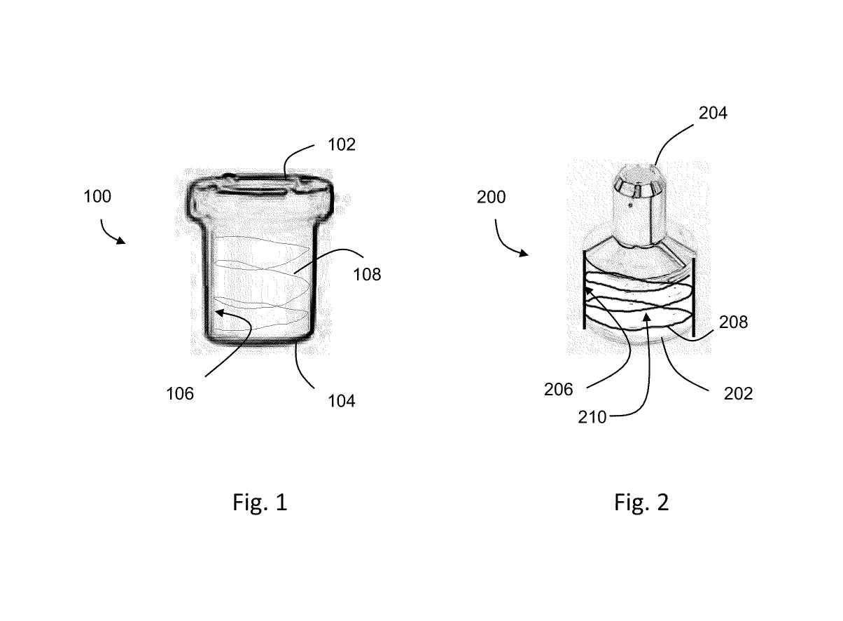

FIG. 1 shows a threaded cap for threading a surgical syringe or the like. In general, the cap 100 may include a closed end 102, an open end 104, an interior surface 106, and a spiral cutting edge 108 to rotatably couple to and thread a predetermined syringe. The cap 100 may be formed of plastic, stainless steel, surgical stainless steel, or any other suitable material.

The closed end 102 may in general have any shape, and may include a resilient, elastic interior surface that yields to and forms a seal with a tip of a syringe to which the cap 100 is attached. The open end 104 may include a circular opening or the like to fit over an end of a syringe.

The interior surface 106 may be shaped and sized to correspond to an end of a predetermined syringe. The specific shape of the syringe and the interior surface 106 is not important. A corresponding fit to an end of any general shape may be suitably devised by one of ordinary skill in the art, although the tightness of physical correspondence between the interior surface 106 and the end of the predetermined syringe will depend upon various design factors such as the height of the spiral cutting edge 108 above the interior surface 106, the ability of the closed end 102 to maintain a seal to the syringe, and the desired tenacity of the seal under various handling and use conditions. For example, where the syringe is intended for use in a centrifuge or the like, a stronger seal may be required and the interior surface 106 may accordingly be fashioned to more closely physically match exterior shape of the end of the syringe.

The interior surface 106 may taper from a wider diameter at the open end 104 to a narrower diameter at the closed end 102. Such a taper may usefully permit the cap 100 to accommodate variations in an outside diameter of the end of the predetermined syringe, or may be appropriate to match a correspondingly tapered end of the syringe.

The spiral cutting edge 108 may be configured to rotatably couple to the predetermined syringe, and to thread the syringe in response to rotational coupling of the cap to the end of the predetermined syringe. The spiral cutting edge 108 may include a sharpened edge to physically cut and deform the end of the syringe during coupling. In order to thread the syringe and form a secure coupling thereto, the spiral cutting edge 108 may be formed of a material substantially harder than the material of the predetermined syringe, or more specifically, the end of the predetermined syringe. In this context, “substantially harder” will be understood to mean sufficiently hard for the spiral cutting edge 108 to functionally thread the end of the syringe and form a secure seal therewith. Actual differences in hardness sufficient for this purpose will depend for example upon the hardness of the end of the syringe and the shape of the cutting edge, and a suitable hardness for the cutting edge relative to the syringe may be readily ascertained by one of ordinary skill.

The spiral cutting edge 108 may be formed into the interior of the cap 100, or may be added in the form of a metallic wire or the like affixed to or embedded in the interior surface 106. This latter approach may be particularly useful, for example, where the cap 100 is formed of plastic or other material having similar hardness to the end of the syringe. The spiral cutting edge 108 may form a double helix (e.g., with two concentric spirals) or any other suitable pattern/shape.

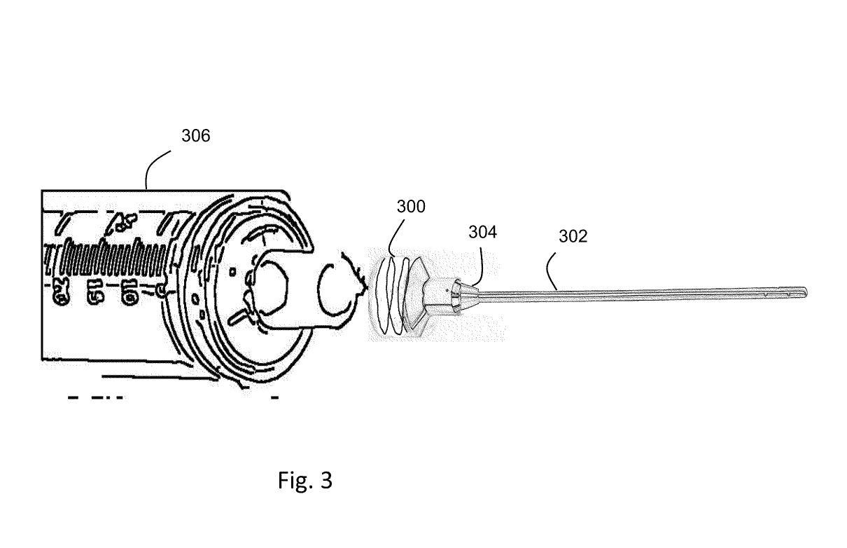

The predetermined syringe, which may for example be the syringe depicted below in FIG. 3, may be a Toomey syringe such as a 60 cc Toomey syringe, or any other syringe suitable for use in a fat transplantation procedure, centrifugation procedure, or any other surgical or similar procedure.

FIG. 2 shows a threaded connector. The connector 200 may be similar in various respects to the cap 100 described above, except that the connector includes an interior lumen and a second opening for transfer of fat or other fluids therethrough from a syringe, e.g., to a transplantation needle. The connector 200 may include a first end 202, a second end 204, an interior surface 206, and a spiral cutting edge 208.

The first end 202 may in general include a circular opening for passage of fat or other fluid therethrough, and may include a circular opening or the like shaped and sized to fit over an end of a predetermined syringe. The second end 204 may be adapted to connect to an injection needle such as an injection needle for a fat transplantation procedure or the like as generally depicted in FIG. 3.

The interior surface 206 may be shaped and sized to correspond to an end of a predetermined syringe. The specific shape of the syringe and the interior surface 206 is not important. A corresponding fit to an end of any general shape may be suitably devised by one of ordinary skill in the art, although the desired or intended tightness of physical correspondence between the interior surface 206 and the end of the predetermined syringe will depend upon various design factors such as the height of the spiral cutting edge 208 above the interior surface 206 and the desired tenacity of the seal under various handling and use conditions.

The interior surface 206 may in general form a lumen 210 that couples the first end 202 to the second end 204 in fluid communication for passage of fat or other material therethrough. The interior surface 206 may taper from a narrower diameter at the first end 202 to a wider diameter and the second end 204. Such a taper may usefully permit the connector 200 to accommodate variations in an outside diameter of the end of the predetermined syringe, or may be appropriate to match a correspondingly tapered end of the syringe.

The spiral cutting edge 208 may be configured to rotatably couple to the predetermined syringe, and to thread the syringe in response to rotational coupling of the cap to the end of the predetermined syringe. The spiral cutting edge 208 may include a sharpened edge to physically cut and deform the end of the syringe during coupling. In order to thread the syringe and form a secure coupling thereto, the spiral cutting edge 208 may be formed of a material substantially harder than the material of the predetermined syringe, or more specifically, the end of the predetermined syringe. In this context, “substantially harder” will be understood to mean sufficiently hard for the spiral cutting edge 208 to functionally thread the end of the syringe and form a secure seal therewith. Actual differences in hardness sufficient for this purpose will depend for example upon the hardness of the end of the syringe and the shape of the cutting edge, and a suitable hardness for the cutting edge relative to the syringe may be readily ascertained by one of ordinary skill.

The spiral cutting edge 208 may be formed into the interior of the connector 200, or may be added in the form of a metallic wire or the like affixed to or embedded in the interior surface 206. This latter approach may be particularly useful, for example, where the cap 100 is formed of plastic or other material having similar hardness to the end of the syringe. The spiral cutting edge 108 may form a double helix (e.g., with two concentric spirals) or any other suitable pattern/shape.

FIG. 3 shows a needle with a threaded connector. In particular, a connector 300 includes an injection needle 302 inserted into a first end 304, and a syringe 306 positioned to insert into and rotationally couple to the connector 300. The syringe 306 may include any of the predetermined syringes described above. As noted, the particular shape and size of the syringe is not important, except inasmuch as the end of the syringe has a predetermined shape and size for which the connector 300 and cutting edges thereof can be suitably arranged and sized.

While particular embodiments of the present invention have been shown and described, it will be apparent to those skilled in the art that various changes and modifications in form and details may be made therein without departing from the spirit and scope of this disclosure and are intended to form a part of the invention as defined by the following claims, which are to be interpreted in the broadest sense allowable by law.

What is claimed is:

1. A device comprising:
a cap having a closed end, an open end, and an interior surface shaped and sized to correspond to an end of a predetermined syringe; and
a spiral cutting edge on the interior surface shaped to thread the end of the predetermined syringe in response to a rotational coupling of the cap to the predetermined syringe, wherein the spiral cutting edge is formed of a material substantial harder than the predetermined syringe.

2. The device of claim 1 wherein the cap is formed of plastic.

3. The device of claim 2 wherein the spiral cutting edge is formed of metal.

4. The device of claim 2 wherein the spiral cutting edge is formed of metallic wire.

5. The device of claim 1 wherein the cap is formed of stainless steel.

6. The device of claim 1 wherein the spiral cutting edge forms a double helix on the interior surface.

7. The device of claim 1 wherein the predetermined syringe is a Toomey syringe.

8. The device of claim 7 wherein the Toomey syringe is a 60 cc Toomey syringe.

9. The device of claim 1 wherein the predetermined syringe is a fat transplantation syringe.

10. The device of claim 1 wherein the interior surface tapers from a wider diameter at the open end to a narrower diameter at the closed end.

11. A device comprising:
a connector having a first end, a second end, a lumen coupling the first end to the second end, and an interior surface shaped and sized to correspond to an end of a predetermined syringe; and
a spiral cutting edge on the interior surface shaped to thread the end of the predetermined syringe in response to a rotational coupling of the first end of the connector to the predetermined syringe, wherein the spiral cutting edge is formed of a material substantially harder than the predetermined syringe.

12. The device of claim 11 further comprising an injection needle coupled to the second end of the connector.

13. The device of claim 12 wherein the injection needle is an injection needle for a fat transplantation procedure.

14. The device of claim 11 wherein the connector is formed of plastic.

15. The device of claim 14 wherein the spiral cutting edge is formed of metal.

16. The device of claim 14 wherein the spiral cutting edge is formed of metallic wire.

17. The device of claim 11 wherein the connector is formed of stainless steel.

18. The device of claim 11 wherein the spiral cutting edge forms a double helix on the interior surface.

19. The device of claim 11 wherein the predetermined syringe is a Toomey syringe.

20. The device of claim 11 wherein the interior surface tapers from a wider diameter at the first end to a narrower diameter at the second end.
